(19) United States
(12) Patent Application Publication
(10) Pub. No.: US 2016/0374124 A1
(43) Pub. Date: Dec. 22, 2016

(54) IMPLANTABLE MEDICAL DEVICE AND METHOD FOR COUPLING AN IMPLANTABLE MEDICAL DEVICE AND AN EXTERNAL DEVICE

(71) Applicant: Biotronik SE & Co. KG, Berlin (DE)

(72) Inventors: Christian GOTHE, Berlin (DE); Thomas FUSS, Berlin (DE)

(73) Assignee: Biotronik SE & Co. KG, Berlin (DE)

(21) Appl. No.: 15/176,755

(22) Filed: Jun. 8, 2016

Related U.S. Application Data
(60) Provisional application No. 62/180,053, filed on Jun. 16, 2015.

(51) Int. Cl. H04W 76/02; H04B 7/24; A61N 1/372; H04B 5/00

(52) U.S. Cl. CPC H04W 76/021; H04B 5/0031; H04B 7/24; A61N 1/37217

(57) ABSTRACT

Embodiments include an implantable medical device (IMD) that includes a control unit, a permanent memory for a device identifier IID, a memory unit that stores a device identifier EID of an external device, a near-field data communication unit and a far-field data communication unit. The control unit, jointly with the near-field data communication unit, detects a third device in the near-field region of the IMD and transmits the device identifier IID of the IMD to the third device. The control unit, following a near-field data communication with a third device, checks any messages received via the far-field data communication unit as to whether the messages include a far-field start message and a device identifier IID of the IMD that corresponds to the device identifier stored in the permanent memory. The control unit sends a far-field response message, which includes the device identifier IID and the device identifier EID.

This application claims the benefit of U.S. Provisional Patent Application 62/180,053, filed on 16 Jun. 2015, the specification of which is hereby incorporated herein by reference.

### BACKGROUND OF THE INVENTION

### Field of the Invention

Embodiments of the invention generally relate to an implantable medical device and a method of coupling an implantable medical device with an external device. In addition, embodiments of the invention generally relate to a system that includes an implantable medical device and an external device.

### Description of the Related Art

Generally, the implantable medical device may be, for example, an implantable heart stimulator, such as a cardiac pacemaker, or an implantable cardioverter/defibrillator (ICD). Devices of this type are typically provided as active devices with an electronic controller, which captures and stores data and/or is programmable. Generally, in order to transmit program data to an implantable medical device of this type or read out captured data from the implantable medical device, an external device is typically provided, which may be a programming device.

Generally, in order to transmit program data to the implantable medical device or in order to transmit data stored in the implantable medical device to the external device, a wireless far-field data communication is typically established between both devices and may be unidirectional or bidirectional. Typically, so that a far-field data communication of this type is not produced randomly between two devices, between which no far-field data communication is intended at a respective moment in time, a respective far-field data communication is preceded by what is known as a pairing, i.e. a coupling, in which the implantable medical device and the external device, which are to communicate with one another by far-field data communication, are assigned to one another for this far-field data communication. Generally, for a pairing of this type, a programming head is typically provided, which is connected by means of a cable to the external device and is used to produce a near-field data communication between the respective implantable medical device and the programming head, in which the implantable medical device and the external device exchange data with one another, the data being suitable for assigning the two devices to one another for the respective far-field data communication.

Generally, in newer programming devices, wireless programming heads are also used instead of the wired programming heads. Typically, a wireless programming head is assigned permanently to a certain programming device by a wireless connection (coupling). With use of a wireless programming head, generally, there is the risk that the programming heads of two programming devices will be mixed up. Typically, a mix-up of the wireless programming heads is then problematic when two programming devices with mixed-up wireless programming heads are in the direct vicinity of one another and are used for aftercare at the same time. As a result of the mix-up, generally, the implant of the adjacent patient may be connected accidentally in such a case to the incorrect programming device during the pairing. In the worst case, typically, the implant of the adjacent patient is thus programmed accidentally.

### BRIEF SUMMARY OF THE INVENTION

One or more embodiments of the invention include a simple and reliable pairing of an implantable medical device and an external device.

At least one embodiment of the invention includes a method of pairing an implantable medical device with an external device. In one or more embodiments, the implantable medical device may be set up for a wireless far-field data communication with the external device and may include a unique device identifier IID. In at least one embodiment, the external device may be set up for a wireless far-field data communication with the implantable medical device and may include a unique device identifier EID. In one or more embodiments, the unique device identifier IID is thus the respective unique device identifier of the implantable medical device, and wherein the device identifier EID is the respective unique device identifier of the external device.

By way of at least one embodiment of the invention, the method may include one or more of the following steps:
near-field transmission of one of the device identifiers, IID or EID, from the implantable medical device or respectively the external device to a third device in the near-field region of the implantable medical device or the external device,
buffering of the respective device identifier IID or EID on the third device,
near-field transmission of the device identifier IID from the third device to the external device or of the device identifier EID from the third device to the implantable medical device, and
initiation of a far-field data communication between the external device with the device identifier EID and the implantable medical device with the device identifier IID.

In one or more embodiments, one of the devices of the system, such as the implantable medical device or the external device, may transmit its own unique device identifier IID or EID to a third device. In at least one embodiment, the third device may be located in the near-field region of the respective device, for example of the implantable medical device or of the external device. In one or more embodiments, the respective unique device identifier, IID or EID, may be buffered in the third device, and the third device may be brought into the vicinity of the other device and therefore into the near-field region thereof. In at least one embodiment, the other device may be the device of which the unique device identifier is not stored on the third device. As soon as the third device is brought into the vicinity of the respective other device, by way of one or more embodiments, the unique device identifier stored in the third device may be transmitted to the other device, such that, in the other device, both its respective own unique device identifier and also the unique device identifier of the first device transmitted via the third device are present.

According to at least one embodiment, the method may include two variants. In one or more embodiments, the first variant may include wherein the third device is first brought into the vicinity and therefore the near-field region of the implantable medical device and receives from the implantable medical device the unique device identifier IID thereof by near-field data communication, the third device buffers this unique device identifier, and then, in the near-field region of the external device, the third device transmits the unique device identifier IID of the implantable medical device to the external device via near-field data communication. As such, in at least one embodiment, both the own unique device identifier EID of the external device and the device identifier IID of the implantable medical device received via the third device are then present in the external device. In one or more embodiments, the external device may then send a far-field start message to the implantable medical device by far-field data communication, wherein the far-field start message may include both the unique device identifier IID of the implantable medical device to which the far-field start message is directed and the unique device identifier EID of the sending external device.

In at least one embodiment of the invention, the second method variant may include wherein the third device is first brought into the near-field region of the external device and receives the unique device identifier EID of the external device by near-field data communication. In one or more embodiments, the unique device identifier EID may be buffered in the third device. In at least one embodiment, the third device may then be brought into the vicinity and therefore into the near-field region of the implantable medical device and may transmit the unique device identifier EID of the external device to the implantable medical device by near-field data communication. In one or more embodiments, both the own unique device identifier IID of the implantable medical device and the unique device identifier EID of the external device received from the third device are then present in the implantable medical device. In at least one embodiment, the implantable medical device may then send a far-field start message, which is directed to the external device of which the unique device identifier EID was received by the implantable device from the third device. In one or more embodiments, the far-field start message may include both the unique device identifier EID of the external device to which the message is directed and the unique device identifier IID of the implantable medical device sending the far-field start message.

By way of one or more embodiments of the invention, the initiation of the far-field data communication may include sending of a far-field start message by the device that has received the unique device identifier EID or IID from the third device, wherein the far-field start message in each case may include both the received device identifier EID or BD and the device identifier BD or EID of the device sending the far-field start message.

According to at least one embodiment of the invention, the unique device identifier IID or EID buffered in the third device may be deleted from the third device once the third device has transmitted this unique device identifier to the other device by near-field data communication. As such, one or more embodiments may ensure that a unique device identifier that may cause an undesired coupling, or an undesired pairing, of external device and implantable medical device does not remain stored on the third device.

In at least one embodiment of the invention, the unique device identifier IID or EID buffered in the third device may be deleted from the third device if the third device has not transmitted this unique device identifier to the other device by near-field data communication within a predefined time. As such, one or more embodiments may ensure that a unique device identifier that may cause an undesired coupling, or an undesired pairing, of external device and implantable medical device does not remain stored on the third device.

In at least one embodiment of the invention, the unique device identifier IID or EID buffered in the third device may be deleted from the third device by the action of a user on the third device. In one or more embodiments, the action may occur by the actuation of a switching device, for example of a button or switch, or the like, on the third device, which causes a deletion of the device identifier IID or EID from the third device. As such, at least one embodiment may ensure that a unique device identifier that may cause an undesired coupling, or an undesired pairing, of the external device and implantable medical device does not remain stored on the third device.

By way of one or more embodiments, the initiation of a far-field data communication may include a sending of a far-field response message by the device that has received the far-field start message and of which the device identifier (IID or EID) is included in the far-field start message. In at least one embodiment, the far-field response message may include the two device identifiers included in the far-field start message, for example the device identifiers of both devices, such as of an implantable medical device and of an external device, which may be coupled via pairing for a far-field data communication.

In one or more embodiments, of the method, the third device in a first step may first be brought into the vicinity and therefore into the near-field region of the external device in order to receive from the external device the unique device identifier EID thereof by near-field data communication and wherein the third device may buffer the unique device identifier. In a second step, in at least one embodiment, the third device may be brought into the vicinity and therefore into the near-field region of the implantable medical device in order to receive from the implantable medical device the unique device identifier IID thereof by near-field data communication, and may buffer the unique device identifier. In one or more embodiments, the unique device identifier EID of the external device stored in the third device may be transmitted in the near-field region of the implantable medical device from the third device to the implantable medical device via near-field data communication. In a third step, in at least one embodiment, the third device may again be brought into the vicinity and therefore into the near-field region of the external device, wherein the third device may transmit the unique device identifier IID of the implantable medical device to the external device via near-field data communication. In one or more embodiment, after these steps, the two device identifiers IID and EID are then present both in the external device and in the implantable medical device. In at least one embodiment, the external device may then send a far-field start message to the implantable medical device by far-field data communication, wherein the far-field start message may include both the unique device identifier IID of the implantable medical device to which the far-field start message is directed and the unique device identifier EID of the sending external device. In one or more embodiments, the implantable medical device to which the far-field start message is directed may check, based on the EID stored thereby in the second step, whether the external device that was provided in the first step for the coupling has sent the far-field start message.

In at least one embodiment, of the method, the third device in a first step may first be brought into the vicinity and therefore into the near-field region of the implantable medical device in order to receive from the implantable medical device the unique device identifier IID thereof by near-field data communication, and may buffer the unique device identifier. In a second step, in one or more embodiments, the third device may be brought into the vicinity and therefore into the near-field region of the external device in order to receive from the external device the unique device identifier EID thereof by near-field communication and may buffer the unique device identifier. In at least one embodiment, the unique device identifier IID of the implantable medical device stored in the third device may be transmitted in the near-field region of the external device from the third device to the external device via near-field data communication. In a third step, in one or more embodiments, the third device may again be brought into the vicinity and therefore into the near-field region of the implantable medical device, wherein the third device may transmit the unique device identifier EID of the external device to the implantable medical device via near-field data communication. After these steps, in at least one embodiment, the two device identifiers IID and EID are then present both in the external device and in the implantable medical device. In one or more embodiments, the implantable medical device may then send a far-field start message to the external device by far-field data communication, wherein the far-field start message may include both the unique device identifier EID of the external device to which the far-field start message is directed and the unique device identifier IID of the sending implantable medical device. In at least one embodiment, the external device to which the far-field start message is directed may check, based on the IID stored thereby in the second step, whether the implantable medical device that was provided in the first step for the coupling has sent the far-field start message.

One or more embodiments of the invention may include a system that includes an implantable medical device, an external device and a third device. In at least one embodiment, the implantable medical device may include a unique device identifier IID and may be set up for wireless far-field data communication with the external device and for near-field data communication with the third device. In one or more embodiments, the external device may include a unique device identifier EID and may be set up for wireless far-field data communication with the implantable medical device and for near-field data communication with the third device. In at least one embodiment, the third device may be set up for near-field data communication with the implantable medical device and the external device, and may receive at least one unique device identifier EID and/or IID from the external device and/or respectively the implantable medical device via near-field data communication, may buffer the at least one unique device identifier and may then transmit the at least one unique device identifier to the other implantable medical device or external device respectively by near-field data communication.

One or more embodiments may include an implantable medical device that includes a control unit, a permanent memory for a unique device identifier IID, a memory unit that stores data and in particular a device identifier EID of an external device, a near-field data communication unit, and a far-field data communication unit. In at least one embodiment, the control unit of the implantable medical device may, in combination with the near-field data communication unit, detect a third device in the near-field region of the implantable medical device and where appropriate may transmit its own device identifier IID to the third device via a near-field data communication with the third device. In one or more embodiments, the control unit of the implantable medical device may, following a near-field data communication with a third device, check any messages received via the far-field data communication unit as to whether the messages include a far-field start message. If the message include a far-field start message, in at least one embodiment, the control unit of the implantable medical device may determine, based on the device identifier IID included in the far-field start message, whether the received far-field start message is intended for this implantable medical device in order to only then transmit into the memory unit of the implantable medical device the device identifier EID, included in the far-field start message, of the external device that has sent the far-field start message. In one or more embodiments, the control unit of the implantable medical device may generate and may send a far-field response message, which includes the device identifier IID of the implantable medical device and also the device identifier EID of the external device stored in the memory unit.

By way of at least one embodiment, the implantable medical device may operate according to the methods discussed herein.

One or more embodiments of the invention may include an implantable medical device that includes a control unit, a permanent memory for a unique device identifier IID, a memory unit that stores data and in particular a device identifier EID of an external device, a near-field data communication unit, and a far-field data communication unit. In at least one embodiment, the control unit of the implantable medical device may, in combination with the near-field data communication unit, detect a third device in the near-field region of the implantable medical device, where appropriate may receive a device identifier EID of an external device via a near-field data communication with the third device, and may store the device identifier in the memory unit. In one or more embodiments, the control unit of the implantable medical device may, following a near-field data communication with a third device, check any messages received via the far-field data communication unit as to whether the messages include a far-field start message and include a device identifier EID of an external device corresponding to the device identifier EID stored in the memory unit. If the messages include the far-field start message and the device identifier EID of an external device, in at least one embodiment, the control unit of the implantable medical device may generate and may send a far-field response message, which includes the device identifier IID of the implantable medical device and also the device identifier EID stored in the memory unit.

By way of one or more embodiments, the implantable medical device may operate according to the methods discussed herein.

In at least one embodiment of the invention, the third device, in addition to or alternatively to the one or more respective device identifiers IID and/or EID, may transmit encryption keys for an encrypted data communication between the implantable medical device and the external device. In one or more embodiments, the transmission may occur parallel to the transmission of the device identifiers EID or IID or in an additional step. If encryption keys are transmitted, in at least one embodiment, the far-field data communication between the implantable medical device and the external device may occur following successful coupling by encrypted data communication. In one or more embodiments, the implantable medical device and the external device may optionally also be coupled by encrypted data communication.

According to at least one embodiment, the encryption keys buffered in the third device may be deleted from the third device once the third device has transmitted these encryption keys by near-field data communication to the other device. As such, one or more embodiments may ensure that no cryptogenic keys remain stored on the third device.

In at least one embodiment, the encryption keys buffered in a third device may be automatically deleted from the third device after a predefined period of time. As such, one or more embodiments may ensure that no encryption keys remain stored on the third device.

At least one embodiment of the invention may include coupling steps, wherein an external device is coupled via the third device with one or more implantable medical devices. In one or more embodiments, the respective pairing may be performed as described herein.

By way of at least one embodiment, the one or more methods as discussed herein include an advantage wherein one or more implantable medical devices may exchange data simultaneously with an external device. Alternatively or additionally, in one or more embodiments, such methods may include an advantage wherein one or more implantable medical devices may be connected simultaneously to a service center via an external device.

At least one embodiment of the invention may include coupling steps, wherein an implantable medical device may be coupled via the third device to one or more external devices. In one or more embodiments, the respective pairing may be performed as described herein.

In at least one embodiment, the implantable medical device may be connected to one or more external devices. In one or more embodiments, a number of situations are possible wherein an implantable medical device may be connected to one or more external devices. At least one embodiment of the invention may include one or more connection scenarios. For example, in one or more embodiments, a connection between an implantable medical device and a patient device, for example a communication device to a service center, may occur, for example for long-term monitoring of the patient, such as home monitoring. In at least one embodiment, the implantable medical device may be connected to a programming device. In one or more embodiments, an implantable medical device may be connected simultaneously to two or more programming devices for training purposes. In at least one embodiment, a connection between an implantable medical device and one or more patient devices, for example communication devices to a service center, may extend the length of time within which an implantable medical device may maintain permanent contact with a service center.

In one or more embodiments, the third device may generate a temporally limited, one-time random value and may transmit the random value to the implantable medical device and the external device in a respective near-field data communication. As such, in at least one embodiment, the temporally limited, one-time random value may be present both in the implantable medical device and in the external device at the moment of the pairing, and may be transmitted to the respective other device within the scope of the far-field start message and/or far-field response message exchanged by far-field data communication. As such, in one or more embodiments, a subsequent far-field data communication may be triggered only when the transmitted, temporally limited, one-time random values match. Should the temporally limited, one-time random values not match, in at least one embodiment, the pairing of the implantable medical device with the external device may be aborted.

In one or more embodiments, the third device may, in the event of a near-field data communication with the implantable medical device and with the external device, initiate in the respective device, prior to the transmission of the IID and/or EID to the third device, the generation of a respective random number by a random number generator in the implantable medical device and/or by a random number generator in the external device. In at least one embodiment, the random number may then be used by the implantable medical device as IID and by the external device as EID and may be transmitted in the existing near-field data communication to the third device. In one or more embodiments, the values thus generated for IID and EID may remain valid until the next near-field data communication with the third device.

In addition to the device identifiers IID and EID or alternatively to the device identifiers IID and EID, in at least one embodiment, a random number for the identification of a far-field data communication may be used as session ID SID. In one or more embodiments, a session ID SID may remain valid until completion of the far-field data communication between an implantable medical device and an external device.

In at least one embodiment, the re-generation of the IID in the implantable medical device or the EID in the external device triggered by the third device may be suppressed when a connection already exists of the implantable medical device to an external device or a connection already exists of the external device to an implantable medical device and a further connection is to be established.

By way of one or more embodiments, an implantable medical device may simultaneously maintain communication paths to one or more external devices or an external device may simultaneously maintain communication paths to one or more implantable medical devices. In at least one embodiment of the invention, a separate IID and/or EID may be used for each communication path.

### BRIEF DESCRIPTION OF THE DRAWINGS

The above and other aspects, features and advantages of at least one embodiment of the invention will be more apparent from the following more particular description thereof, presented in conjunction with the following drawings, wherein:

FIG. 1 shows an overview of a system comprising an implantable medical device, an external device and a third device;

FIGS. 2 to 9 show method steps for coupling the implantable medical device and the external device via the third device;

FIG. 10 shows a schematic illustration of the implantable medical device with components thereof;

FIG. 11 shows a schematic illustration of the external device with components thereof; and

FIG. 12 shows a schematic illustration of the third device and components thereof.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

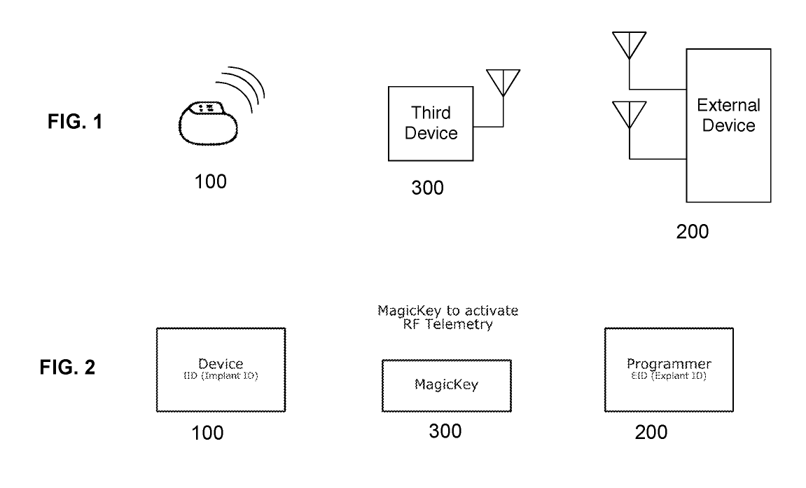

FIG. 1 shows a system that includes an implantable medical device 100, an external device 200 and a third device 300, according to one or more embodiments of the invention. In at least one embodiment, the implantable medical device 100 may be or may include, for example, an implantable cardiac pacemaker, an implantable neurostimulator, an implantable drug pump or an implantable cardioverter/defibrillator (ICD). In one or more embodiments, the external device 200 may be a programming device. In at least one embodiment, the third device may be or may include an auxiliary device that couples the implantable medical device 100 to the external device 200.

In one or more embodiments, the implantable device 100 may be set up such that the implantable device 100 may communicate by far-field data communication with the external device 200 and may communicate by near-field data communication with the external device 300. In at least one embodiment, the external device 200 may be set up such that the external device 200 may communicate with the implantable medical device 100 by far-field data communication and may communicate with the third device 300 by near-field data communication. In one or more embodiments, the third device 300 may communicate both with the external device 200 and with the implantable medical device 100 by near-field data communication.

In at least one embodiment, near-field data communication may be or may include a short-range electromagnetic data communication, such as NFC, an inductive data communication such as coil telemetry, of programming devices, or an acoustic data communication in the audible or ultrasound range. In one or more embodiments, the near-field data communication may include or may be a data communication such that the two devices communicating with one another are to be located in the direct vicinity of one another.

By way of at least one embodiment, far-field data communication may be or may include a data communication such as via medical implant communication service (MICS), Bluetooth, medical Bluetooth, WLAN, ZigBee or the like, which allows for a greater distance between the devices communicating with one another. In one or more embodiments, the greater distance and therefore the range of the far-field data communication may be, for example, a few meters, and wherein the near-field data communication may include a range from a few centimeters to at most a meter.

In the method steps illustrated in FIGS. 2 to 9, according to one or more embodiments of the invention, the external device 200 is designated as “Programmer”, the implantable medical device 100 is designated as “Device” and the third device 300 is designated as “Magic Key”.

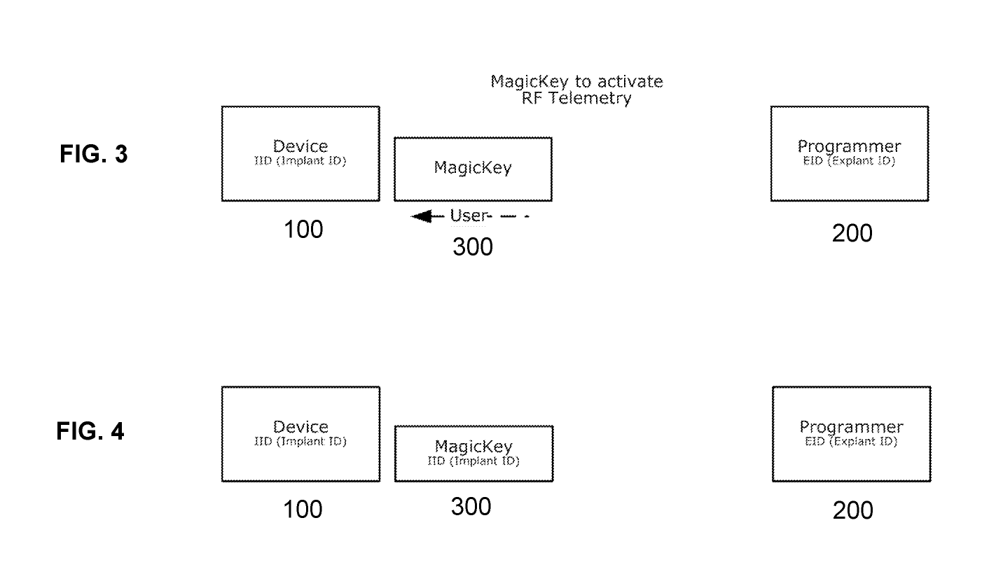

In at least one embodiment of the invention, FIGS. 2 to 9 illustrate a method of coupling the implantable medical device 100 with the external device 200, wherein the third device 300 may first be brought into the vicinity of the implantable medical device 100; as shown in FIG. 3. In one or more embodiments, a near-field data transmission may then occur between the implantable medical device 100 and the third device 300, wherein the implantable medical device 100 may transmit its own unique device identifier IID to the third device 300; as shown in FIG. 4. In at least one embodiment, the unique device identifier IID of the implantable medical device 100 may then be stored in the third device 300; as shown in FIG. 4.

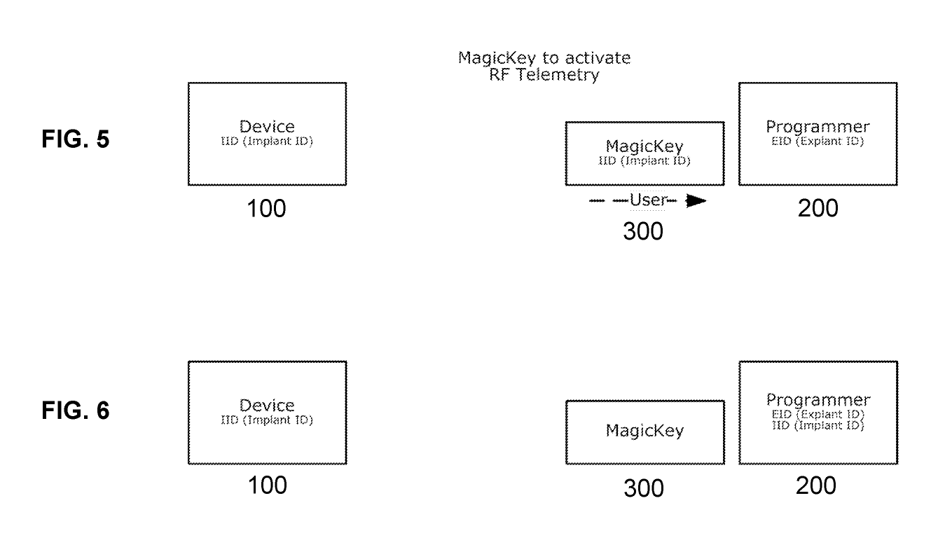

In a next step, according to one or more embodiments of the invention, the third device 300 may then be brought into the vicinity of the external device 200; as shown in FIG. 5.

In at least one embodiment, the unique device identifier IID of the implantable medical device 100 buffered in the third device 300 may then be transmitted to the external device 200. In one or more embodiments, the transmission may occur via the near-field data communication; as shown in FIG. 6. In at least one embodiment, a far-field data communication may now begin.

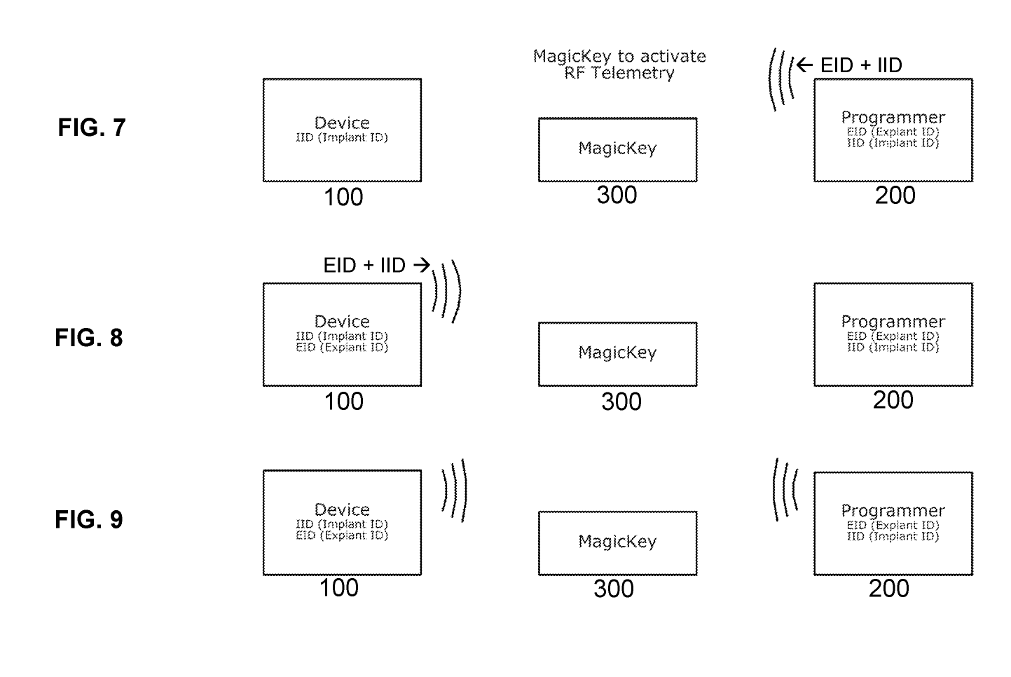

In order to initiate the far-field data communication, by way of at least one embodiment, the external device 200 may send a far-field start message via far-field data communication to the implantable medical device 100. In one or more embodiments, the far-field start message may include both the unique device identifier EID of the external device 200 and the unique device identifier IID of the implantable medical device 100; as shown in FIG. 7. In at least one embodiment, the implantable medical device 100 may check whether a far-field start message received thereby includes the device identifier IID belonging to the implantable medical device. If the message includes the device identifier IID, in one or more embodiments, the implantable medical device 100 may generate and may transmit a far-field response message to the external device 200. In at least one embodiment, the generation and transmission may occur via the far-field data communication. In one or more embodiments, the far-field response message may include the device identifier IID of the implantable medical device and also the device identifier EID of the external device 200; as shown in FIG. 8.

In at least one embodiment, the far-field response message may be received by the external device 200. In one or more embodiments, the external device 200 may then compare the device identifiers EID and IID received with the far-field response message with its own device identifier EID and with the device identifier IID of the implantable medical device 100 that was previously received by the external device 200 from the third device 300 via near-field data communication. If the check of these device identifiers IID and EID received via the far-field response message via far-field data communication reveals a match with the device identifiers EID and IID stored in the external device 200, in at least one embodiment, the coupling may be completed successfully and a further unidirectional or bidirectional far-field data communication for the exchange of data, for example program data, between the external device 200 and the implantable medical device 100 may occur; as shown in FIG. 9.

At least one embodiment of the invention may ensure that both device identifiers IID and EID are known both to the implantable medical device 100 and to the external device 200 already before the initialization of the far-field data communication.

In at least one embodiment of the invention, for example of a method of coupling the implantable medical device 100 to the external device 200, the third device 300 may first be brought into the vicinity of the external device 200. In one or more embodiments, the third device 300 may optionally already be located at the external device 200, for example wherein the third device 300 may be or may include a pen, such as a stylus, to operate the external device 200. In at least one embodiment, the third device 300 may first receive a unique device identifier EID of the external device 200. In one or more embodiments, this may occur via a near-field data communication between the external device 200 and the third device 300. In at least one embodiment, the unique device identifier EID of the external device 200 may then be buffered in the third device 300.

In one or more embodiments, the third device 300 may then be brought with the buffered device identifier EID of the external device 200 into the vicinity of the implantable medical device 100.

In at least one embodiment, a near-field data transmission may then take place between the implantable medical device 100 and the third device 300, in which the third device 300 transmits the unique device identifier EID of the external device 200 to the implantable medical device 100. In one or more embodiments, the implantable medical device 100 may transmit its own unique device identifier IID via near-field data transmission to the third device 300. In at least one embodiment, both the unique device identifier EID of the external device 200 and the unique device identifier IID of the implantable medical device 100 may then be stored in the third device 300.

In a next step, in one or more embodiments, the third device 300 may then be brought again into the vicinity of the external device 200.

In at least one embodiment, the device identifiers buffered in the third device 300, specifically at least the unique device identifier IID of the implantable medical device 100, may then be transmitted to the external device 200. In one or more embodiments, the transmission may occur via the near-field data communication. In at least one embodiment, the unique device identifier EID of the external device 200 may be transmitted to the external device 200. In one or more embodiments, the external device 200 may check whether the device identifier EID transmitted from the third device 300 by near-field data communication to the external device 200 actually corresponds to the device identifier EID of the respective external device 200. If the device identifies EID responds, by way of at least one embodiment, the assignment of external device 200 and implantable medical device 100 is positive and a far-field data communication may begin.

In order to initiate the far-field data communication, according to one or more embodiments, the external device 200 may send a far-field start message via far-field data communication to the implantable medical device 100. In at least one embodiment, the far-field start message may include both the unique device identifier EID of the external device 200 and the unique device identifier IID of the implantable medical device 100. In one or more embodiments, the implantable medical device 100 may check whether a far-field start message received thereby includes the device identifier IID belonging to the implantable medical device and the device identifier EID of the external device 200 that was previously transmitted via near-field data communication from the third device 300 to the implantable device 100. If the message does include the device identifier IID belonging to the implantable medical device and the device identifier EID of the external device 200, in one or more embodiments, the implantable medical device 100 may generate and may transmit a far-field response message to the external device 200. In at least one embodiment, the generation and transmission may occur via the far-field data communication. In one or more embodiments, the far-field response message may include the device identifier IID of the implantable medical device 100 and also the device identifier EID of the external device 200.

In at least one embodiment, the far-field response message may be received by the external device 200. In one or more embodiments, the external device 200 may then compare the device identifiers EID and IID received with the far-field response message with the own device identifier EID of the external device 200 and with the device identifier IID of the implantable medical device 100 received by the external device 200 previously from the third device 300 via near-field data communication. If the check of these device identifiers IID and EID received via the far-field response message via far-field data communication reveals a match with the device identifiers EID and IID stored in the external device 200, by way of at least one embodiment, the coupling is successfully completed and a further unidirectional or bidirectional far-field data communication for the exchange of data, for example program data, between the external device 200 and the implantable medical device 100 may occur.

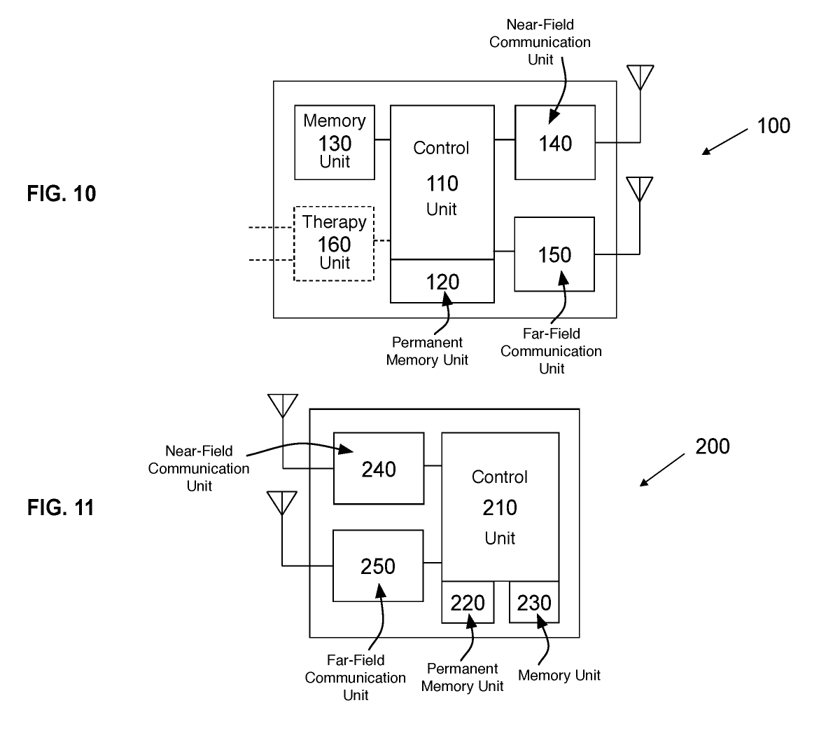

FIG. 10 shows in a schematic illustration components of the implantable medical device 100, according to one or more embodiments of the invention. In at least one embodiment, the components may include a control unit 110 of the implantable medical device, which is connected to a permanent memory 120 of the implantable medical device, and which includes a unique device identifier IID of the implantable medical device 100. In one or more embodiments, the control unit 110 of the implantable medical device may be connected to a memory unit 130 of the implantable medical device, in which memory unit program data or data recorded by the implantable medical device 100 and also data received via data communication may be included. In at least one embodiment, the memory unit 130 of the implantable medical device may be used to store any received device identifier EID of an external device used to pair the implantable medical device and the external device.

In one or more embodiments, the control unit 110 of the implantable medical device may be connected to a near-field data communication unit 140 of the implantable medical device and a far-field data communication unit 150 of the implantable medical device. In at least one embodiment, the near-field data communication unit 140 of the implantable medical device may be used for near-field data communication with the third device 300, whereas the far-field data communication unit 150 of the implantable medical device may be used for the further data exchange between the implantable medical device and, for example, an external device 200.

In one or more embodiments, the control unit 110 of the implantable medical device may be connected to one or more therapy or monitoring units 160 of the implantable medical device, for example stimulation or sensing units of a cardiac pacemaker.

FIG. 11 shows the external device 200 with components thereof, according to one or more embodiments of the invention, including a control unit 210 of the external device, which is connected to a permanent memory 220 of the external device, and which includes the device identifier EID of the external device 200 discussed herein. In at least one embodiment, the control unit 210 of the external device may be connected to a memory unit 230 of the external device, in which program data and also data received via data communication, for example data recorded by the implantable medical device 100, may be included. In one or more embodiments, the memory unit 230 of the external device may be used to store any received device identifier IID of an implantable medical device used to pair the implantable medical device and the external device. In at least one embodiment, the control unit 210 of the external device may be connected to a near-field data communication unit 240 of the external device and also to a far-field data communication unit 250 of the external device. In one or more embodiments, the near-field data communication unit 240 of the external device may be used for the near-field data communication with the third device 300, whereas the far-field data communication unit 250 of the external device may be used for other data communication, for example to transmit programming data from the external device 200 to the implantable medical device 100 or for receiving data recorded by the implantable medical device 100.

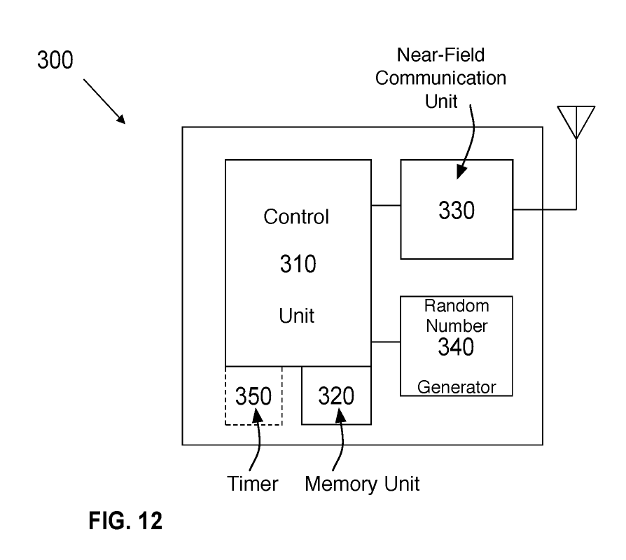

FIG. 12 shows the third device 300 and components thereof, according to one or more embodiments of the invention. In at least one embodiment, the components may include a control unit 310 of the third device and a memory unit 320 of the third device, wherein the memory unit may be connected to the control unit. In one or more embodiments, the memory unit 320 of the third device may be used to temporarily store device identifiers IID and EID of the implantable medical device 100 or respectively of the external device 200, wherein the identifiers were received by near-field data communication, and may temporarily store the encryption key to be transmitted and the currently valid random number.

In at least one embodiment, the control unit 310 of the third device may be connected to a near-field data communication unit 330 of the third device, via which a near-field data communication both with the external device 200 and with the implantable medical device 100 may occur, depending on whether the third device 300 is located in the vicinity of the external device or the implantable medical device.

Depending on the used components, by way of one or more embodiments, the third device 300 may be actively or passively operated. In at least one embodiment of the invention, the third device 300 may include energy memory units, such as capacitors, batteries or accumulators, not shown in FIG. 12, for example to perform energy management.

In one or more embodiments, the third device 300 may include a random number generator 340, which generates a temporally limited, one-time random number. In at least one embodiment, the third device 300 may then transmit the temporally limited, one-time random number to the implantable medical device 100 and the external device 200 in a respective near-field data communication.

Via the far-field start message and/or the far-field response message, by way of one or more embodiments, the respective random number may be transmitted as discussed herein, and the far-field data communication may be continued only when the value matches on both devices.

In at least one embodiment, the third device 300 may include a timer 350 connected to the control unit 310. In one or more embodiments, the timer 350 of the third device may be used by the control unit 310 to remove from the third device 300 data stored in the memory unit 320 of the third device after a predefined or predefinable time. In at least one embodiment of the invention, the data may be or may include, for example, one or more of random numbers, the device identifier EID of the external device, the device identifier IID of the implantable medical device, encryption keys and other information stored in the short term on the third device used to pair the implantable medical device and the external device.

In one or more embodiments, the third device 300 may include a switching device, such as a switch or a button, which may be connected to the control unit 310. In at least one embodiment, the actuation of the switching device may be used to remove from the memory unit 320 of the third device data stored in the third device. For example, in one or more embodiment, the data stored may include one or more of random numbers, the device identifier EID of the external device, the device identifier IID of the implantable medical device, encryption keys and other information stored in the short term on the third device used to pair the implantable medical device and the external device.

One or more embodiments of the invention present various advantages.

For example, in at least one embodiment, third device 300 may be one or more of
wireless,
small,
light, and
disinfectable.

For example, in at least one embodiment, the method includes
intuitive operation, and
a more convenient apparatus than use of a programming head.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

100 implantable medical device
110 control unit of the implantable medical device
120 permanent memory of the implantable medical device
130 memory unit of the implantable medical device
140 near-field data communication unit of the implantable medical device
150 far-field data communication unit of the implantable medical device
160 therapy or monitoring units of the implantable medical device
200 external device
210 control unit of the external device
220 permanent memory of the external device
230 memory unit of the external device
240 near-field data communication unit of the external device
250 far-field data communication unit of the external device
300 third device
310 control unit of the third device
320 memory unit of the third device
330 near-field data communication unit of the third device
340 random number generator of the third device
350 timer of the third device

What is claimed is:

1. A method of pairing an implantable medical device with an external device, wherein the implantable medical device comprises wireless far-field data communication to communicate with the external device and a unique device identifier IID, and wherein the external device comprises a wireless far-field data communication to communicate with the implantable medical device and a unique device identifier EID, the method comprising:
near-field transmission of the IID from the implantable medical device or of the EID from the external device to a third device in a near-field region of the implantable medical device or the external device,
buffering of the respective unique device identifier IID or EID on the third device,
near-field transmission of the unique device identifier IID from the third device to the external device or of the device unique identifier EID from the third device to the implantable medical device, and
initiation of a far-field data communication between the external device with the unique device identifier EID and the implantable medical device with the unique device identifier IID.

2. The method according to claim 1, further comprising deleting the unique device identifier IID or EID from the third device once the third device has transmitted the unique device identifier IID or EID to the external device or respectively to the implantable medical device.

3. The method according to claim 1, wherein the initiation of a far-field data communication comprises sending a far-field start message by the implantable medical device or the external device that has received the unique device identifier EID or IID from the third device, wherein the far-field start message comprises both the received unique device identifier EID or IID and the unique device identifier IID or EID of the implantable medical device or of the external device sending the far-field start message.

4. The method according to claim 3, wherein the initiation of a far-field data communication further comprises sending a far-field response message by the implantable medical device or the external device that has received the far-field start message and of which the unique device identifier IID or EID is included in the far-field start message, wherein the far-field response message comprises the two unique device identifiers IID and EID included in the far-field start message.

5. The method according to claim 1, further comprising transmitting an encryption key via the third device, and wherein the far-field data communication between the implantable medical device and the external device is an encrypted data communication following a successful coupling between the implantable medical device and the external device.

6. The method according to claim 1, further comprising coupling the external device via the third device with one or more implantable medical devices.

7. The method according to claim 1, wherein the third device
generates a temporally limited one-time random value, and
transmits the random value to the implantable medical device and the external device in a respective near-field data communication,
such that the temporally limited one-time random value is
present both in the implantable medical device and in the external device while pairing the implantable medical device and the external deice, and
transmitted to the respective other device via the far-field start message and/or far-field response message exchanged by far-field data communication, wherein a subsequent far-field data communication is triggered only when the transmitted temporally limited one-time random values match.

8. The method according to claim 1, wherein the third device
is brought from the external device to the implantable medical device, such that the third device buffers the unique device identifier EID of the external device,
transmits the unique device identifier EID of the external device to the implantable medical device,
receives from the implantable medical device the unique device identifier IID,
buffers the unique device identifier IID,
brought again into a vicinity of the external device, and
transmits the unique device identifier IID of the implantable medical device to the external device.

9. An implantable medical device (100) comprising:
a control unit (110),
a permanent memory (120) that stores a unique device identifier IID of the implantable medical device (100),
a memory unit (130) that stores a unique device identifier EID of an external device (200),
a near-field data communication unit (140), and
a far-field data communication unit (150),
wherein the control unit (110) is configured, in combination with the near-field data communication unit (140), to
detect a third device (300) in the near-field region of the implantable medical device (100) and
transmit the unique device identifier IID to the third device (300) using a near-field data communication with the third device (300), and
wherein the control unit (110) is configured, following a near-field data communication with the third device (300) via the far-field data communication unit (150), to check any received messages as to whether the messages comprise a far-field start message,
wherein when the messages comprise a far-field start message, the control unit (110) is further configured to determine, based on the unique device identifier IID included in the far-field start message, whether the received far-field start message is intended for the implantable medical device (100), and
wherein when it is determined that the received far-field start message is intended for the implantable medical device (100), the control unit (110) is further configured to
transmit into the memory unit (130) of the implantable medical device (100) the unique device identifier EID included in the far-field start message of the external device (200) that sent the far-field start message, and
generate and send a far-field response message, wherein the far-field response message comprises the unique device identifier IID of the implantable medical device (100) and the unique device identifier EID stored in the memory unit (130).

10. The implantable medical device (100) according to claim 9, wherein
the control unit (110) is further configured, in combination with the near-field data communication unit (140) following the detection of the third device (300) in the near-field region of the implantable medical device (100), to
receive the unique device identifier EID of the external device via near-field data communication with the third device and to store the unique device identifier EID in the memory unit (130), and,
the control unit (110) is further configured to check, following a near-field data communication with the third device, any messages received via the far-field data communication unit (150) as to whether the messages comprise a far-field start message and the unique device identifier EID of the external device corresponding to the unique device identifier EID stored in the memory unit (130) and,
wherein if the messages comprise a far-field start message and the unique device identifier EID of the external device corresponding to the unique device identifier EID stored in the memory unit (130), the control unit is further configured to generate and to send a far-field response message, which comprise the unique device identifier IID of the implantable medical device (100) and also the unique device identifier EID stored in the memory unit (130).

11. A system comprising:
an implantable medical device (100),
an external device (200) and
a third device (300),
wherein the implantable medical device (100) comprises a unique device identifier IID and comprises wireless far-field data communication to communicate with the external device (200) and comprises near-field data communication to communicate with the third device (300),
wherein the external device (200) comprises a unique device identifier EID, wireless far-field data communication to communicate with the implantable medical device (100) and near-field data communication to communicate with the third device (300), and
wherein the third device (300) comprises near-field data communication to communicate with the implantable medical device (100) and the external device (200), and
wherein the third device (300) is configured to
receive one or more of at least one unique device identifier EID from the external device and at least one unique device identifier IID from the implantable medical device (100) via near-field data communication,
buffer one or more of the at least one unique device identifier EID and IID, and
transmit one or more of the at least one unique device identifier EID and IID to the other device of the implantable medical device or the external device respectively, by near-field data communication.

12. The system according to claim 11, wherein the third device (300) is further configured, in the event of a near-field data communication with the implantable medical device (100) and with the external device (200), to
initiate in the respective device, prior to the transmission of one or more of the IID and EID to the third device, a generation of a respective random number by one or more of a random number generator in the implantable medical device (100) and a random number generator in the external device (200),
wherein the random number is used by the implantable medical device (100) as the IID and by the external device (200) as the EID,
wherein the random number is transmitted in the near-field data communication to the third device (300), and
wherein the IID and EID each remain valid until a next near-field data communication with the third device (300).

13. The system according to claim 11, wherein the third device (300) further comprises a random number generator (340), wherein the random number generator (340) generates a temporally limited one-time random number, and wherein the third device (300) is further configured to transmit the temporally limited one-time random number in a respective near-field data communication to the implantable medical device (100) and to the external device (200).

14. The system according to claim 11, wherein the third device (300) is an operating pen used with the external device (200).

15. The system according to claim 11, wherein the third device (300) is further configured to transmit an encryption key, wherein the far-field data communication between the implantable medical device (100) and the external device (200) is an encrypted data communication following a successful coupling between the implantable medical device (100) and the external device (200).
